(19) United States
(12) Patent Application Publication
(10) Pub. No.: US 2002/0098183 A1
(43) Pub. Date: Jul. 25, 2002

(54) Anti-selectin antibodies for prevention of multiple organ failure after polytrauma and for prevention of acute organ damage after extracorporeal blood circulation

(76) Inventors: Ulrich Martin, Munchen (DE); Anton Haselbeck, Weilham (DE); Gunther Schumacher, Bernried (DE); Man Sung Co, Cupertino, CA (US)

Correspondence Address: HOWREY SIMON ARNOLD & WHITE, LLP, Box 34, 301 Ravenswoods Avenue, Menlo Park, CA, 95025, US

(21) Appl. No.: 09/917,410

(22) Filed: Jul. 26, 2001

Related U.S. Application Data
(63) Continuation of application No. 09/013,871, filed on Jan. 27, 1998.
Application No. 09/013,871 is a continuation of application No. PCT/US96/13152, filed on Aug. 16, 1996.
Application No. PCT/US96/13152 is a continuation-in-part of application No. 08/578,953, filed on Dec. 27, 1995, now abandoned.

(51) Int. Cl. A61K 39/395

(52) U.S. Cl. 424/137.1

(57) ABSTRACT

Anti-selectin antibodies for reducing probability of incidence of polytraumatic events, such as organ failure.

### FIELD OF THE INVENTION

This invention relates to the use of anti-selectin antibodies for the prevention of multiple organ failure associated with polytrauma and for the prevention of acute organ damage associated with extracorporeal blood circulation. Especially preferred are antibodies to E-selectin, L-selectin, and/or P-selectin.

### BACKGROUND OF THE INVENTION

A polytrauma is understood as an injury of a number of tissues (bones or soft tissues). In a polytraumatic event, mediator systems (e.g. cytokines, arachidonic acid products, oxygen radicals, proteases) as well as leukocytes and other cells are activated. This can lead to secondary organ damage (e.g. destruction of tissue structures by liberated proteases). This secondary organ damage can occur in the whole body independently of the site of the primary trauma.

A polytrauma may also be associated with hemorrhagic shock. Hemorrhagic shock is understood as a shock which is characterized by a rapid and substantial loss of blood toward the inside or outside. At present, hemorrhagic shock can be treated successfully by intensive medical therapy, especially by volume substitution and blood transfusion. The combination of hemorrhagic shock and trauma is referred to as hemorrhagic-traumatic shock. In contrast to pure hemorrhagic shock, there is, at present, no specific therapy for traumatic or hemorrhagic-traumatic shock and no prophylaxis at all for later organ failure following polytrauma.

Multiple organ failure (MOF) is a severe problem which often occurs after polytraumas. The more organs affected, the higher the mortality. Organs and systems which can fail include the heart, lung, kidney, liver, stomach, intestinal system and central nervous system. Although in recent years it has been possible to reduce the very high mortality of trauma patients to about 20% by improvements in rescue service and emergency medicine, so far there is no specific therapy for organ failure.

Is Marzi et al., J. Trauma 35: 110-119 (1993) discloses that superoxide dimutase can be given 24 hours after trauma. However, the results are not unequivocal and merely show a trend towards a partial improvement. A substantial reduction in mortality and MOF was, however, not observed. Mileski, W. J. et al., Surgery 108: 206-212 (1990) discloses that the binding of neutrophils or their aggregation contributes substantially to the development of hemorrhagic shock after organ damage. In this case, experimental animals were given anti-CD18 antibodies immediately after a 90 minute phase of hemorrhagic shock. Therapeutic methods for treatment of multiple organ failure after polytrauma is not described by Mileski. Vedder N. B. et al., Surgery 106: 509-516 (1989) also propose the use of anti-CD18 antibodies to treat hemorrhagic shock.

Selectins, such as L, E, and P-selectin, have been found to be associated with tissue damage during the course of ischemia and reperfusion. Neutrophils play an important role in this connection. It is assumed that selectin is required for the recruitment of neutrophils. Apparently L-selectin is necessary for the complete development of damage in skeletal muscle as well as in the lung (Seekamp A. et al., Am. J. Pathol. 11: 592-598 (1994). Mulligan, M. S. et al., J. Immunol. 151: 832-840 (1994) describe a similar phenomenon.

The production of humanized anti-L-selectin antibodies is described in WO 94/12215, incorporated herein by reference. The use of such antibodies in the treatment of inflammatory diseases and in particular of myocardial infarction is proposed. A dose of 1-50 mg is proposed to prevent acute lung failure. However, the reference does not describe a method for preventing MOF after polytrauma.

Thus, there is a need for effective treatment of preventing and/or treating multiple organ failure after polytrauma.

Acute organ damage can also be caused during cardiovascular surgery, such as an aorto-coronary vein bypass operation or a cardiac valve operation, where the blood of the patient circulates extracorporeally through a heart-lung machine. The extent of the damage depends upon the period during which the machine is in operation. This can lead, e.g., to failure of the lungs, which can necessitate artificial respiration of the patient well after the operation (Birnbaum, D. et al., Z. Kardiol. 79, Suppl. 4: 87-93 (1990)). Other organs, such as the heart, kidneys, liver or systems such as the blood and coagulation system may also be damaged and fail.

It is known from Mulligan, M. S. et al., J. Immunol. 151: 832-840 (1994) that molecules which promote adhesion such as L, E, and P-selectins are involved in acute inflammatory processes. These molecules mediate the adhesive interaction of leukocytes with endothelial cells. In this connection L-selectin seems to play an important role in the initial phase (rolling) of acute intrapulmonary inflammatory reactions. Mulligan states further that anti-L-selectin antibodies are suitable for shortening the duration of the lung damage that can be triggered by L-selectin.

However, up to now no preventive therapy is known which can be used to prevent acute organ damage that is caused by extracorporeal circulation of the blood. Thus, there is a need for effective treatment of preventing acute organ damage caused by extracorporeal circulation of the blood.

### OBJECTS OF THE INVENTION

An object of the invention is to provide a method and a therapeutic composition which can be used to effectively prevent multiple organ failure after polytrauma in humans and to considerably reduce the mortality rate of polytrauma patients. The invention concerns the use of anti-selectin antibodies therapeutically and for the production of pharmaceutical compositions useful in the prevention of multiple organ failure and death after polytrauma.

An object of the invention is also to provide a method and use of a therapeutic composition which contains anti-selectin antibodies for the prevention of acute organ damage after extracorporeal circulation. Such organ damage can be largely avoided with this method and this procedure. A particular advantage of the method is the extracorporeal application which leads to an effective decrease in organ complications.

### BRIEF DESCRIPTION OF THE FIGURES

FIG. 1 shows the lung wet weight of experimental animals with respect to the observation time.

FIG. 2 shows the cardiovascular parameter CO (cardiac output) with respect to time for the various experimental animals.

FIG. 3 shows the cardiovascular parameter MAP (mean arterial blood pressure) with respect to time for the experimental animals.

FIG. 4 shows the BE value (arterial base excess) with respect to time for the experimental animals.

FIG. 5 shows the number of white blood cells with respect to time for the various experimental animals.

### DETAILED DESCRIPTION OF THE INVENTION

The invention concerns the use of at least one anti-selectin antibody for the production of a pharmaceutical composition to prevent acute organ damage after extracorporeal circulation of a patient's blood through a heart-lung machine, wherein 1 to 30 minutes before ending the extracorporeal circulation the anti-selectin antibody is added extracorporeally into the tube system of the heart-lung machine at a dose of 1.0-10 mg/kg of body weight of the patient and preferably 2-4 mg/kg.

Surprisingly, acute organ damage after extracorporeal circulation of a patient's blood can be prevented to a large extent by this preventive extracorporeal administration. In a preferred embodiment a total of 1-3 further doses of 1-4 mg/kg anti-selectin antibody, such as an anti-L-selectin antibody, are administered to the patient within 1-3 days. Polyclonal or monoclonal, murine, human, chimeric or humanized antibodies/immunoglobulins and their binding fragments can be used as the anti-selectin antibodies. In one aspect of the invention, the therapeutic compositions are not administered into the body of the patient, but extracorporeally, i.e., directly into the tube system of a heart-lung machine.

“Anti-selectin antibodies”, as used herein, refers to any antibody which binds to a selectin. Especially preferred are antibodies which bind specifically to one of L-selectin, E-selectin, or P-selectin, as well as combinations of these. Also preferred are antibodies which react with more than one selectin, such as antibodies which react with both L- and E-selectin. L-selectin is a known glycoprotein that is constitutively expressed by all leukocytes. Both L-selectin and its murine homologues, GP90 and Mell4, are involved in the normal recirculation of lymphocytes—each mediates the interaction between circulating lymphocytes and vascular ligands (often referred to as “addressins”) on the high endothelial venules (HEVs) of lymphoid organs (L. A. Lasky, et al., Cell 69: 927-938 (1992); E. L. Berg, et al., J. Cell. Biol. 114: 343-349 (1991)). In addition to its role as a lymphocyte homing receptor, L-selectin is also involved in the adhesion of circulating leukocytes to non-lymphoid tissues, such as endothelium, during inflammation. L-selectin is shed from the leukocyte surface following leukocyte activation (T. K. Kishimoto, et al., Science 245: 1238-1241 (1989)), and this may be an important process in retaining activated leukocytes at sites of inflammation. L-selectin has an amino-terminal carbohydrate-recognition domain (CRD) that has considerable homology with C-type lectins (K. Trickhamer, J. Biol. Chem. 263 9557-9560 (1988)), followed by a single epidermal-growth-factor-like domain, complement-regulatory domains, a single transmembrane polypeptide and a carboxy-terminal cytoplasmatic domain. L-selectin interacts with its cognate ligand through the amino-terminal CRD in a calcium dependent manner.

In accordance with the invention, anti-selectin antibodies are preferred which modulate, and more preferably inhibit, the interaction between the CRD domain and the corresponding carbohydrate receptors on the surface of cells. Such carbohydrate receptors are described by R. B. Parekh, Tibtech 12: 339-345 (1994), incorporated by reference. These carbohydrate receptors may be phosphorylated or sulfated sugars.

In a further embodiment of the invention, anti-P- and/or anti-E-selectin antibodies are used instead of, or in addition to, anti-L-selectin antibodies. Such antibodies can be produced using P- or E-selectin (described in R. B. Parekh and T. F. Tedder, FASEB Journal 9: 866-873 (1995), incorporated by reference). In an especially preferred embodiment of the invention, anti-P- and/or anti-E-selectin antibodies are used which show considerable cross-reactivity with L-selectin antibodies, especially cross-reactivity with antibody HuDreg-55 or HuDreg-200.

As used herein, the term “humanized immunoglobulin” refers to an immunoglobulin comprising a human framework, at least one complementarity determining region (CDR) from a non-human antibody, and in which any constant region present is substantially identical to a human mmunoglobulin constant region, i.e., at least about 85-90%, preferably at least 95% identical. Hence, all parts of a humanized immunoglobulin, except possibly the CDRs, are substantially identical to corresponding parts of one or more native human immunoglobulin sequences. For example, a humanized immunoglobulin would not encompass a chimeric mouse variable region/human constant region antibody. See, e.g., European Patent Application EP A 451216, incorporated by reference

The invention also concerns the use of such anti-selectin antibodies to reduce MOF and mortality after polytrauma. It has surprisingly turned out that it is possible to prevent multiple organ failure. when anti-selectin antibodies, especially anti-L-selectin antibodies, are administered very soon after the polytrauma. This is also surprising because there are no acute symptoms at this early stage and there would therefore have been no reason to administer such a dose as a preventive measure.

It also has surprisingly turned out that anti-selectin antibodies in doses of 1.0-10 mg/kg, preferably of 2-4 mg/kg, administered one to five times, preferably once or twice after the polytraumatic event can advantageously be used, where the first application is given as early as possible, preferably 0.5-8 hours, and especially preferably, 0.5-4 hours after the polytraumatic event. The intervals between the individual applications are between about 6 and about 72 hours, preferably between 6 and 36 hours.

In a preferred embodiment the dose and time of the second and subsequent preventive applications is selected depending on the concentration of the anti-selectin antibodies in the blood and preferably in plasma or serum, which is an early determinable parameter. In this connection it is preferable that the plasma concentration of the anti-selectin antibody is maintained at 10-100 μg/ml over a time period of 7-10 days after the polytraumatic event. This concentration is equivalent to about a 10-100 fold excess over the concentration of soluble selectin in plasma. The dose and time for the second and subsequent applications are determined by determining the concentration of the anti-selectin antibody in blood, serum or plasma at intervals of 6-24 hours and immediately administering a dose which essentially corresponds to the dose of the first application when the plasma concentration falls below 10 μg/ml antibody. When the antibody concentration is between 10 and 50 μg/ml, the antibody is administered at about half the concentration of the first application, and at an antibody concentration between 50 and 100 μg/ml, no further antibody is administered. In this case only the antibody concentration is monitored further.

The anti-selectin antibody concentration in blood, serum or plasma is determined by the usual methods, preferably by an immunological method of determination. Such methods are known to a person skilled in the art. For example the determination can be carried out by means of an ELISA test in which a labelled selectin specific antibody, preferably the antibody which is also used therapeutically, competes for a specific selectin. In a subsequent step the amount of labelled antibody which has bound to the antigen is then determined and the concentration of the anti-selectin antibody in the sample is determined from this.

The therapeutic compositions of the invention are usually administered parenterally such as intravenously, intraarterially, intraperitoneally, subcutaneously or intramuscularly. Intravenous (i.v.) administration is preferred. The active components of the composition can be used in a liquid or solid form, preferably in a lyophilized form and be used together with a suitable diluent or carrier such as water or aqueous solutions of sodium chloride, dextrose, buffers and so forth. Other suitable pharmaceutical auxiliary substances can also be added.

Antibodies to selectin are known from the state of the art and are described for example in EP-A 0 386 906, WO 93/00111 and WO 94/12215 and by Kishimoto, T. K. et al., in Blood 78: 805-811 (1991) and Proc. Natl. Acad. Sci. USA 87: 2241-2248 (1990), all of which are incorporated by reference. L-selectin is also denoted LECAM-1, Mel 14 or Lam-1 in the literature. The cloning and sequence of Lam-1 have been described in WO 93/02698. Antibodies which bind specifically to selectin are suitable. Humanized antibodies, especially HuDreg 200 which is described in WO 94/12215 and is expressly incorporated herein by reference, are suitable. Other antibodies which bind to selectin, such as HuDreg 55, (sequence: SEQ ID NO: 1-4), are also particularly preferred.

“Antibody” as used herein is understood as a protein that is composed of one or several polypeptide chains which are essentially encoded by antibody genes. The antibody genes code for the antigen-specific variable regions and may also code for the genes for the constant regions. Antibodies within the sense of the invention are also understood as various derivatives and fragments of antibodies such as Fv, Fab and F(ab)₂ and individual antibody chains (Houston et al., PNAS USA 85 5879-5883 (1988), Bird et al., Science 242: 423-426 (1988), Hood et al., Immunology, Benjamin N.Y., 2nd edition (1984), Hunkapiller and Hood, Nature 323 15-16 (1986)). Monoclonal antibodies and fragments thereof are preferably used and particularly preferably chimeric or humanized antibodies preferably of the IgG1 or IgG4 subtype.

The antibodies preferably contain at least two light polypeptide chains and two heavy polypeptide chains. Each of these chains contains a variable region (usually the N-terminal part of the polypeptide chain) which in turn contains a domain which binds the antigen. Heavy and light chains additionally contain a constant region of the polypeptide (usually the C-terminal part) which mediates the binding of the antibody to leukocytes (neutrophils, lymphocytes etc.). Usually the light and heavy chains are complete antibody chains which are composed of the variable region and the complete constant region. In this connection, the variable regions and the constant regions can be derived from different antibodies, for example different isotypes. For example a polypeptide which contains the variable region of a heavy chain of an anti-selectin antibody of the γ-1 isotype may be linked to the constant region of the heavy chain of an antibody from another class (or subclass).

Anti-selectin antibodies are also suitable in which one or several amino acids are substituted. In this case, amino acids are preferably substituted by other amino acids with similar characteristic features (e.g. the acidic amino acid Asp by the acidic amino acid Glu). The structural characteristics of the original sequence are not changed by such substitutions. Examples of such polypeptide structures are described in Proteins, Structures and Molecular Principles, Creighton (editor), W. H. Freeman and Company, New York (1984); Introduction to Protein Structure, C. Brandon and J. Tooze, Garland Publishing, New York (1981); Thornton et al., Nature 354 105 (1991). In general, antibodies which are suitable as anti-selectin antibodies are those which bind to one or more of L-selectin, E-selectin, and P-selectin and/or inhibit the rolling of leukocytes (e.g. neutrophils).

In addition to the humanized immunoglobulins specifically described herein, other “substantially homologous” modified immunoglobulins can be readily designed and manufactured utilizing various recombinant DNA techniques well known to those skilled in the art. Human antibodies, including, for example, the Eu or GAL antibodies, as well as other human antibodies known in the art, can be used as a source of framework sequence. These framework sequences should exhibit a high degree of sequence identity with the mouse Dreg 55 or mouse Dreg 200 variable region domains from which the CDRs were derived. The heavy and light chain variable framework regions can be derived from the same or different human antibody sequences. Indeed, the heavy and light chain framework regions can each be derived from more than one human antibody. The human antibody sequences can be the sequences of naturally occurring human antibodies or can be consensus sequences of several human antibodies. See Carter et al., WO 92/22653 (1992), incorporated by reference.

“Antibodies which are capable of binding in an equivalent manner” are understood as those antibodies which bind to the same or an overlapping epitope of a selectin. Epitope overlap can be determined by methods known in the art, for example with the aid of a competitive test system. A competitive binding assay may be carried out for this and the extent to which the antibody competes with, e.g., HuDreg 55 for binding to an immobilized L-selectin antigen is determined. For this, L-selectin immobilized in a suitable manner (preferably L-selectin on leukocytes) is incubated with HuDreg 55 in a labelled form and an excess of the antibody to be tested. The extent of the binding of the antibody to be tested to L-selectin is determined in comparison to HuDreg 55 by determining the bound label of the anti-leucocyte-bound label. If the labelled HuDreg 55 is displaced by at least 50% by the antibody to be tested an epitope overlap is present. Antibodies that bind in an equivalent manner as HuDreg 55 are preferred for use in the invention.

“Antibodies which are capable of binding in an equivalent manner” can also be identified by screening for the capacity to block neutrophil-endothelial cell interaction. A simple visual assay for detecting such interaction has been described by Kishimoto et al. (Blood, 78:805 (1991)). Briefly, monolayers of human umbilical vein cells are stimulated with interleukin-1. Neutrophils, with or without pretreatment with the antibody under test, are added to the monolayer under defined conditions, and the number of adhering neutrophils is determined microscopically. Ir. one method, the neutrophils are obtained from human leukocyte adhesion deficient patients. See Anderson et al., Ann. Rev. Med. 38:175 (1987). The neutrophils from such patients lack integrin receptors, whose binding to neutrophils might obscure the effects of blocking L-selectin binding.

The antibodies can be used as complete monoclonal antibodies, fragments thereof (Fv, (Fv)₂, Fab′, F(ab′)₂), chimeric, humanized or human antibodies. Short antibody fragments which only contain the CDR regions or parts thereof which bind specifically to L-selectin can also be used.

The production of antibodies and in particular of monoclonal antibodies and fragments thereof is familiar to a person skilled in the art and described for example in E. Harlow and D. Lane, Antibodies: A Laboratory Manual, Cold Spring Harbor Press (1988), Bessler et al., Immunobiol. 170: 239-244 (1985), Jung et al., “Angewandte Chemie” 97: 883 (1985), Cianfiglia et al., Hybridoma Vol. 2: 451-457 (1993).

Anti-selectin antibodies that can be used according to the invention can also be produced by recombinant means. Such processes are described in Sambrook et al., Molecular Cloning: A Laboratory Manual, 2nd edition (1989), Cold Spring Harbor, New York, Berger and Kimmel, Methods in Enzymology, Vol. 152, Guide to Molecular Cloning Techniques (1987), Academic Press Inc., San Diego Calif., which are incorporated by reference. Such recombinant antibodies can be produced either in eukaryotic or prokaryotic cells by processes known to the art. Mammalian cells, especially lymphocytic cell lines, are preferably used as host cells. Chimeric, humanized or human antibodies are preferably produced by recombinant methodalogue. Regions can be selected for the non-antigen binding regions of the antibodies which are for example described in E. A. Kabat et al., Sequences of Proteins of Immunological Interest (1987), National Institute of Health, Bethesda Md. The production of recombinant anti-L-selectin antibodies of humanized and human antibodies is described in WO 94/12215, which is hereby incorporated by reference. A particularly preferred, humanized anti-L-selectin antibody is HuDreg 55, which may be constructed in the same manner as HuDreg 200 described therein, and comprises two light chains having the sequence SEQ ID NO: 2 and two heavy chains having the sequence SEQ ID NO: 4.

Preferred humanized immunoglobulins are those which bind to selectin with a binding affinity of at least 1×10⁷ M⁻¹ in standard binding conditions (e.g., phosphate-buffered saline with 2 percent fetal bovine serum at 25° C.). Examples of such humanized immunoglobulins are HuDreg 55 and HuDreg 200. More preferred are humanized antibodies, which preferably bind, in standard binding conditions, to human selectin with an affinity of at least 1×10⁸ M⁻¹, and more preferably, with an affinity of at least 1×10⁹ M⁻¹, and advantageously with an affinity of at least 1×10¹⁰ M⁻¹ or more. Usually, the binding affinity of a humanized immunoglobulin is within a factor of 3-10 of the mouse immunoglobulin from which it was derived. For example, the affinity of the mouse Dreg 200 antibody is about 10⁸ M⁻¹ and that of mouse Dreg 55 is about 10⁹ M⁻¹.

The following examples, sequence protocols, publications and figures further elucidate the invention. The described processes are to be understood as examples of illustration, not of limitation, which also after modifications, describe the subject matter of the invention.

### EXAMPLE 1

Use Of Anti-L-Selectin Antibody To Reduce Post-Trauma Organ Failure

The protective action of a humanized antibody against L-selectin (anti-L-selectin) in reducing post-traumatic organ failure such as that which typically occurs after injury in patients with severe polytrauma is demonstrated. Humanized anti-L-selectin antibody (HuDreg 55) is used as the antibody. It also reacts with baboon L-selectin. This mouse form of this antibody is described by Kishimoto PNAS, USA 87 (1990) 2244-2248. The humanized sequence is shown in SEQ ID NO: 1-4.

The HuDreg 55 and HuDreg 200 antibodies react with L-selectin on human leukocytes; however, only HuDreg 55 reacts with L-selectin of baboon leukocytes. Therefore HuDreg 55 was used. Since HuDreg 55 and HuDreg 200 bind in the same concentration range to human leukocytes (e.g. in FACS analysis), the effects of HuDreg 55 on baboons is presumptively equivalent to the effect of HuDreg 200.

As a model, severe tissue damage with associated hypovolemia (=loss of liquid and blood towards the inside and/or outside) was induced in baboons. The pure blood loss with subsequent shock (hemorrhagic shock) is less relevant for the lung damage (Pretorius et al., J. Trauma 1987; 27: 1344-1353; Schlag et al., page 384-402, in Schlag, Redl: Pathophysiology of Shock, Sepsis, and Organ Failure, Springer Verlag, Berlin, 1993). This is in agreement with clinical experience which shows that lung complications only occur very rarely in pure hemorrhagic shock (Schlag et al., 1993 see above).

In order to determine the frequency and severity of post-traumatic lung failure it was necessary to observe the animals (named: SELEC 971, SELEC 979 (treated); and Co 968, Co 969, Co 970 (control)) over several days; however, for ethical reasons, it was not possible to induce bone fractures in conscious animals and leave them untreated for several days so that in this subchronic model the tissue trauma is simulated. The activation of the complement system appears to be the earliest trigger for the activation of cellular systems and plays a key role in the rapid occurrence of a non-bacterial inflammatory reaction of the body (Schlag et al., 1993, supra). Therefore, in the model, complement was activated by cobra venom factor.

The mortality for multiple organ failure after severe polytrauma is given as 15-30% in the relevant literature. In the present animal model the severity of the polytrauma was increased to the extent that the mortality is at least twice as high and occured earlier than in humans. Therefore the observation period was limited to three days.

Adult baboons with a body weight (BW) between 18 and 22 kg were admitted to the study after three months quarantine. The fasted animals were sedated with ketamine (6-8 mg/kg), subsequently intubated and attached to a CPAP respirator (continuous positive airway pressure) (inspiratory O₂ concentration of 25±2%). The anesthesia was maintained with 1-3 mg/kg/h pentobarbital. The animals breath spontaneously. A Swan Ganz catheter was pushed forward into the pulmonary artery via the right femoral vein. A catheter for withdrawing blood and measuring blood pressure was tied into the right arm artery. A large lumen catheter is introduced into the femoral artery for the temporary collection of blood. A catheter for infusions, medication and blood collection was introduced into the left arm vein. The bladder was catheterized for the measurement of urine production. The Swan Ganz catheter and the arterial catheter were left for three days. For fluid balance the animals received 5 ml/kg/h Ringer solution (electrolyte solution for parenteral liquid substitution) during the anaesthetic phase. The blood temperature of the animals was kept at ≧37° C. with the aid of an infrared lamp. Blood gas analyses were carried out (pO₂, pCO₂, pH, BE, HCO₃—) and hemodynamic parameters were determined (MAP, RAP, PAP, CO, HR). Lung function was determined by means of the respiratory rate (RR) and end expiratory CO₂. Blood samples were collected repeatedly in order to measure the number of white blood cells (WBCs). Cobra venom factor was administered at a dose of 10 U/kg per i.v. at the beginning of the retransfusion and administered again at a dose of 5 U/kg 1 hour after beginning the retransfusion of the blood. The blood withdrawal for triggering the hypovolemia was regulated such that the MAP (mean arterial pressure) came to lie between 40 and 50 mm Hg and the CO (cardiac output) is reduced by 50 to 70%. Approximately 50 ml/kg blood were usually withdrawn for this and stored until retransfusion. The deficient circulation was maintained for two to three hours and was controlled in such a way that the base excess was no more than −5 to −7 mEq. At the end of this shock phase retransfusion of the previously collected blood was begun. This phase lasted 4 hours.

The retransfusion was complemented by an additional administration of Ringer solution. Humanized antibody HuDreg 55 or the corresponding volume of saline solution as a placebo was administered intravenously 15 minutes after the start of retransfusion. Anti-L-selectin antibody was administered at a dose of 2 mg/kg. At the end of retransfusion the animals were awakened from anaesthesia and were returned to their cages for observation. At times 24 h, 48 h and 72 h a low level of anesthesia was again induced and the measuring parameters were registered and blood was withdrawn. If the animals had not died before the end of the three day observation period, they were then sacrificed and autopsied. The main terminal points of the study were mortality, survival period and organ damage, for example, to the lung.

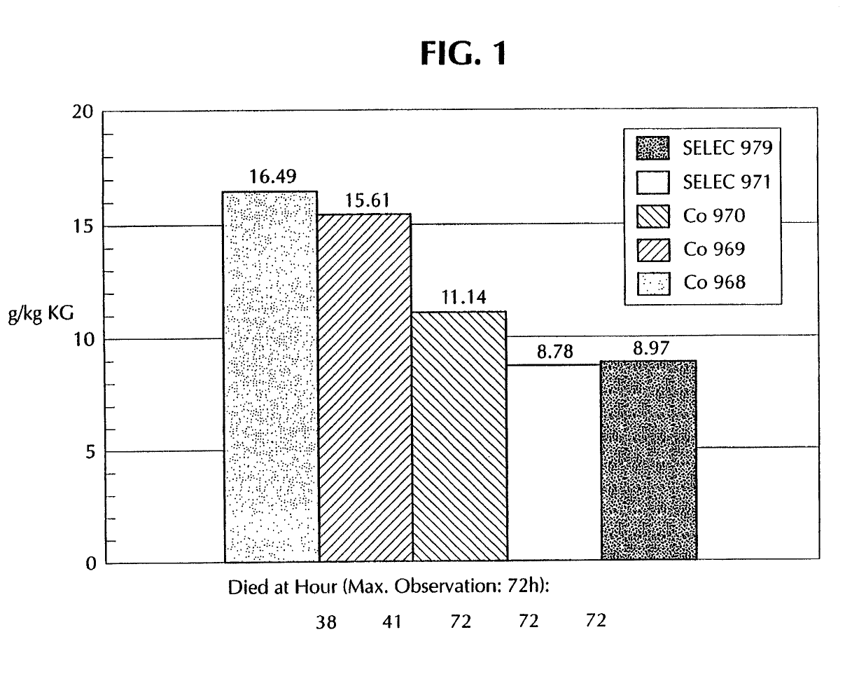

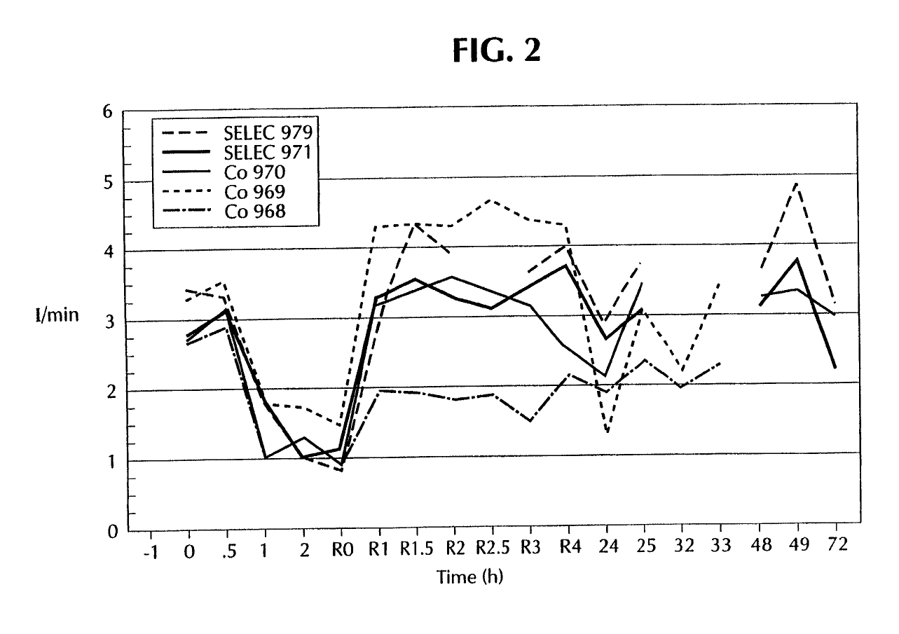

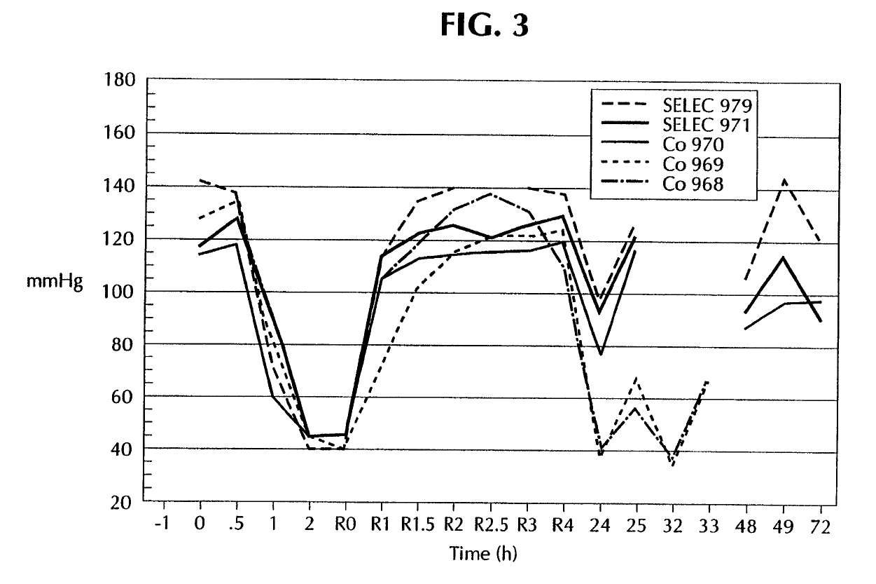

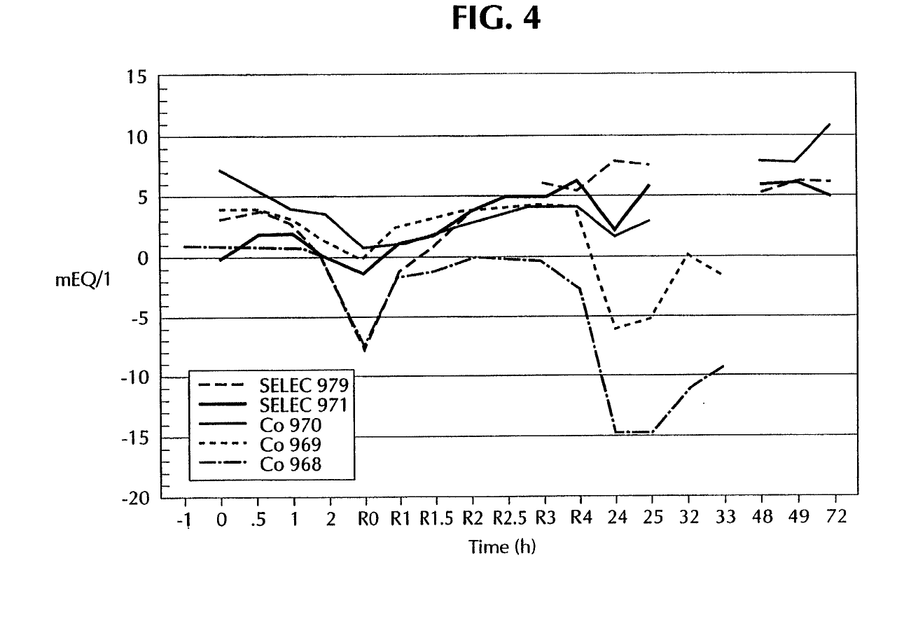

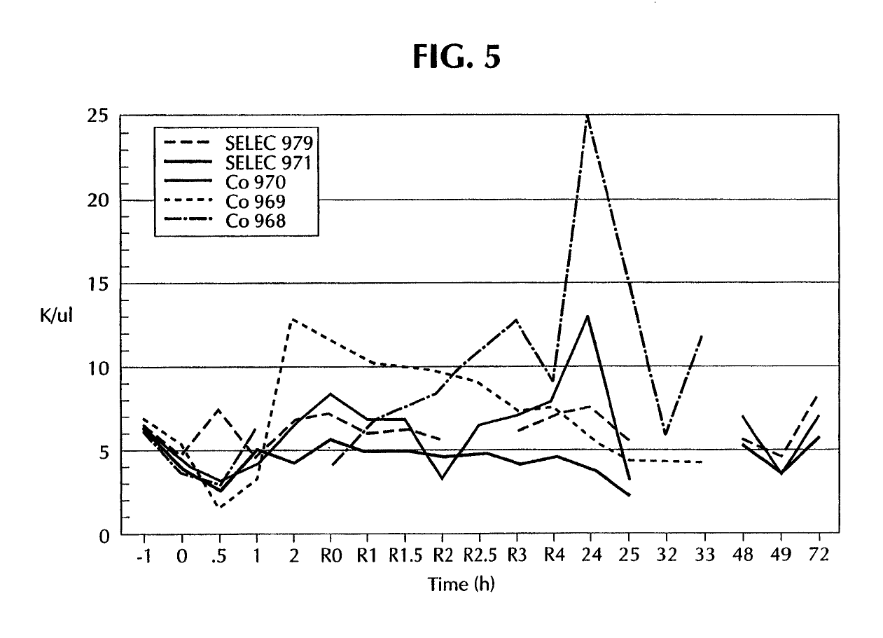

In the first experiment, three control animals were treated with placebo solution and two with the HuDreg 55 humanized antibody. Of the three control animals, two died before the end of the three day observation period at 38 h and 41 h whereas both anti-L-selectin treated animals survived. The lung wet weight, as an expression of organ damage, was almost normal in the antibody treated animals (normal values 7-8 g/kg BW) whereas it had increased considerably in all three placebo-treated control animals (FIG. 1). This is due to infiltration of fluid after the permeability disorder. The cardiovascular parameters Co₂ and MAP (FIG. 2 and 3) are better at 24 hours in the surviving animals than in the control animals. The dying control animals also have a negative arterial base excess (BE) indicating a disturbed acid-base balance (FIG. 4). The leucocytosis (increase in white blood cells) observed in the control animals is absent in the antibody animals (FIG. 5).

### EXAMPLE 2

Use Of Anti-L-Selectin Antibody To Reduce Post-Traumatic Mortality

The experiments reported in Example 1, supra, were continued and expanded to include 28 baboons which were randomly assigned to one of two experimental groups conducted as described in Example 1. The baboons received either 2 mg/kg i.v. of anti-L-Selectin antibody or the appropriate placebo volume-dose as control 15 minutes after initiation of reperfusion after the ischemia period. The main endpoints for statistical analysis of the study were mortality at the end of the 3-day observation period and survival time. Fisher's exact test was used for mortality analysis and the log-rank-test was used for survival time analysis. One-sided p-values (reduction of mortality or prolongation of survival time by active treatment) are reported. The null hypothesis was rejected only when the probability (p) of the calculated statistic was p<0.05.

Anti-L-selectin antibody reduced (p<0.05) mortality from 10 out of 14 (=71%) baboons in the control group to 3 out of 14 (=21%) baboons in the active treatment group at a level of statistical significance. In addition, survival time in the anti-L-Selectin group was prolonged to 64.4 h, whereas animals in the control group died earlier (p<0.05), on an average at 42.1 h. This difference was statistically significant.

The table summarizes the results:

| | Mortality | Survival time (h) |
|---|---|---|
| Anti-L-Selectin | 3/14* | 64.4 ± 4.7* |
| Antibody | | |
| Placebo-control | 10/14 | 42.4 ± 5.7 |

Mean ± standard error of mean;
n = 14 per group;
*, p < 0.05 by Fisher's exact test;
⁺, p < 0.05 by log-rank-test.
Observation period was 72 h.

These data show that early treatment of baboons suffering from ischemia-reperfusion injuries due to hemorrhgaic-traumatic shock with administration of anti-L-Selectin significantly prolongs survival time and reduces mortality as compared to placebo-control.

### EXAMPLE3

Use of Anti-L-Selectin Antibody To Reduce Organ Damage After Extracorporeal Blood Circulation

The protective action of a humanized antibody against L-selectin, preferably HuDreg 55, in reducing organ damage after extracorporeal blood circulation such as that which typically occurs after long operating periods of the heart-lung machine in cardiac surgery was studied.

As a model, severe lung damage was caused in baboons by letting the heart-lung machine, which takes over the function of the lungs and heart after the heart is stopped, run for several hours. After the machine was turned off, the pumping action of the heart was resumed, and endogenous circulation and respiration restarted, massive infiltration of activated leukocytes into the pulmonary circulation caused severe damage to the lungs. The leukocytes present in the pulmonary circulation locally release toxic mediators at a high concentration which led to damage of the vascular endothelium with subsequent increase in permeability. In this process fluid crossed over from the vascular space into the alveoli (smallest pulmonary alveoli) which led to an accumulation of fluid in the lung. This impeded gas exchange in the lung and artificial respiration becomes necessary. The oxygen demand increased as the impairment in gas exchange increases in severity and this was further aggravated by a fibroproliferative transformation of the alveolo-endothelial barrier. Thus, in particularly severe cases, the concentration of inhaled oxygen in the respiratory air which is usually about 20% has to be increased to about 100%. Nevertheless, in such cases, the supply of pure oxygen is insufficient to maintain the arterial oxygen concentration or oxygen partial pressure in the blood (paO2) at an adequate level.

The fibroproliferative transformation process and pulmonary edema result in an increase in the pressures in the arteria pulmonalis which is connected to the lung and this leads to a strain on the right heart. If these reactions build up further this finally leads to death by heart-lung failure.

Adult baboons with a body weight (BW) between 18 and 22 kg are admitted to the study after three months quarantine. The fasted animals were sedated with ketamine (6-8 mg/kg), intubated, and attached to a CPAP respirator (inspiratory O₂ concentration of 25±2%). The anesthesia was maintained with 1-3 mg/kg/h pentobarbital. The animals breathed spontaneously. A Swan Ganz catheter was pushed forward into the pulmonary artery via the right femoral vein. A catheter for withdrawing blood and measuring blood pressure was tied into a right arm artery. A catheter for infusions, medication and blood collection was introduced into a left arm vein. The bladder is catheterized to measure the production of urine. For fluid balance the animals receive 5 ml/kg/h Ringer solution. The temperature of the animals is maintained at 37° C. with the aid of an infrared lamp. Blood gas analyses are carried out (pO₂, pCO₂, pH, BE, HCO₃—) and hemodynamic parameters are determined (MAP, RAP, PAP, CO, HR). The lung function is determined by means of the respiratory rate (RR) and end expiratory CO₂. Blood samples are collected repeatedly in order to measure the number of white blood cells (WBC).

At the start of the experiment the thorax was opened (thoracotomy) and the vena cava and the aorta was prepared. Afterwards, first the vena cava and then the aorta were cannulated so that blood from the vena cava flowed into the heart-lung machine and later back into the aorta. A peristaltic pump assumes the pumping function of the heart in the heart-lung machine and ensures maintenance of the pressure gradient required for circulation. Exchange of oxygen and binding of carbon dioxide is achieved by membrane oxygenation. The blood was heparinized so that the tubes and blood vessels do not get blocked. The blood flows back to the aorta via the tube system and is distributed in the body via the normal vascular system.

The heart-lung machine takes over the function of the heart and lung. The heart is stopped while the machine is in operation so that the operating surgeon can for example work on the cardiac valves (insert prostheses).

Fifteen minutes before the end of the four hour extra-corporeal circulation, a dose of 2 mg/kg HuDreg 55 or the same volume dose of placebo was administered directly into the tube system of the heart-lung machine. The animal was observed for a further four hours after ending the extracorporeal circulation. Measurements are carried out repeatedly before, during and after the extra-corporeal circulation. In particular arterial blood gases and parameters for acid-base balance are recorded, cardiovascular parameters such as the mean arterial blood pressure, right atrial pressure, pulmonary artery pressure, cardiac output and heart rate are determined, the lung function is measured (e.g. end expiratory CO₂) and blood samples are withdrawn for hematological, clinical-chemical (e.g. kidney and liver function) and biochemical analyses. In addition, urine production (kidney function) is measured. Further, parameters for permeability disorders in the lung were determined. At the end of the experiment, the animals were sacrificed and necropsy and histological examinations were carried out in order to determine the degree of damage to the various organs and systems such as heart, lung, liver, kidney, intestine, CNS, blood etc. It is expected that the animals treated with HuDreg 55 sustain less organ damage than those treated with placebo.

What is claimed is:

1. A method for reducing multiple organ failure of a patient after a polytraumatic event, comprising administering an amount of an anti-L-selectin antibody in a pharmaceutically acceptable carrier to said patient, in the amount sufficient to reduce said multiple organ failure, wherein the first dose of said anti-L-selectin antibody is administered from 0.5 to 8 hours after said polytraumatic event.

2. The method according to claim 1, wherein the first dose of said anti-L-selectin antibody is administered from 0.5 to 4 hours after said polytraumatic event.

3. The method according to claim 1, wherein the first dose of said anti-L-selectin antibody is administered before said patient has any acute symptoms of said multiple organ failure.

4. The method of claim 1, wherein the anti-L-selectin antibody is humanized.

5. The method of claim 4, wherein the anti-L-selectin antibody is HuDreg 55 or HuDreg 200.

6. The method of claim 4, wherein anti-L-selectin antibody comprises a light chain comprising an amino acid sequence as set forth in SEQ If) NO: 2 and a heavy chain comprising an amino acid sequence as set forth in SEQ ID NO: 4.

7. The method of claim 4, wherein anti-L-selectin antibody comprises a light chain comprising an amino acid sequence as set forth in SEQ ID NO: 5 and a heavy chain comprising an amino acid sequence as set forth in SEQ ID NO: 6.

8. A method for prevention of multiple organ failure of a patient after a polytraumatic event, comprising administering an amount of an anti-L-selectin antibody in a pharmaceutically acceptable carrier to said patient, in the amount sufficient to prevent said multiple organ failure, wherein the first dose of said anti-L-selectin antibody is administered from 0.5 to 8 hours after said polytraumatic event.

9. The method according to claim 8, wherein the first dose of said anti-L-selectin antibody is administered from 0.5 to 4 hours after said polytraumatic event.

10. The method of claim 8, wherein the anti-L-selectin antibody is humanized.

11. The method of claim 10, wherein the anti-L-selectin antibody is HuDreg 55 or HuDreg 200.

12. The method of claim 10, wherein the anti-L-selectin antibody comprises a light chain comprising an amino acid sequence as set forth in SEQ ID NO: 2 and a heavy chain comprising an amino acid sequence as set forth in SEQ ID NO: 4.

13. The method of claim 10, wherein the anti-L-selectin antibody comprises a light chain comprising an amino acid sequence as set forth in SEQ ID NO: 5 and a heavy chain comprising an amino acid sequence as set forth in SEQ ID NO: 6.

14. A method for treating a patient who has suffered a severe polytraumatic event, comprising administering to said patient a therapeutically effective amount of anti-L-selection antibody in a pharmaceutically acceptable carrier to said patient, wherein the first dose of said anti-L-selectin antibody is administered from 0.5 to 8 hours after said polytraumatic event, wherein the first dose of said anti-L-selectin antibody is administered before said patient has any acute symptoms of multiple organ failure resulting from said severe polytraumatic event.

15. The method according to claim 14, wherein the first dose of said anti-L-selectin antibody is administered from 0.5 to 4 hours after said severe polytraumatic event.

16. The method of claim 14, wherein the anti-L-selectin antibody is humanized.

17. The method of claim 14, wherein the anti-L-selectin antibody is HuDreg 55 or HuDreg 200.

18. The method of claim 17, wherein the anti-L-selectin antibody comprises a light chain comprising an amino acid sequence as set forth in SEQ ID NO: 2 and a heavy chain comprising an amino acid sequence as set forth in SEQ ID NO: 4.

19. The method of claim 17, wherein the anti-L-selectin antibody comprises a light chain comprising an amino acid sequence as set forth in SEQ ID NO: 5 and a heavy chain comprising an amino acid sequence as set forth in SEQ ID NO: 6.


SEQUENCE LISTING (6 sequences)

SEQ ID NO: 1 - DNA (cDNA), 654 bp; double-stranded, linear
  CDS 1,,654
gacattcagatgacccaatctccgagctctttgtctgcgtctgtaggggatagggtcactatcacctgcaaggccagccaaagtgttgattatgatggtgatagttatatgaactggtaccaacagaaaccaggaaaggcacccaagcttctcatctatgctgcatccaacctagaatctggtatcccatccaggtttagtggcagtgggtctgggacagacttcaccctcaccatctcttctctgcagccggaggatttcgcaacctattactgtcagcaaagtaatgaagatccgtggacgttcggtcaaggcaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 2 - protein, 218 aa
DIQMTQSPSSLSASVGDRVTITCKASQSVDYDGDSYMNWYQQKPGKAPKLLIYAASNLESGIPSRFSGSGSGTDFTLTISSLQPEDFATYYCQQSNEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA (cDNA), 1329 bp; double-stranded, linear
  CDS 1,,1329
  mat_peptide 1
gaagtgcaactggtggagtctgggggaggcttagtgcagcctggaggaagcttgagactctcctgtgcagcctctggattcactttcagtacctatgccatgtcttgggttcgccaggctccagggaagggactcgagtgggtcgcatccattagtactggtggtagcacctactatccagacagtgtgaagggccgattcaccatctccagagataatgccaagaacaccctgtacctgcaaatgaattctctgagggctgaggacacggccgtgtattactgtgcaagagactatgacgggtattttgactactggggccaaggcaccctggtcacagtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 4 - protein, 443 aa
EVQLVESGGGLVQPGGSLRLSCAASGFTFSTYAMSWVRQAPGKGLEWVASISTGGSTYYPDSVKGRFTISRDNAKNTLYLQMNSLRAEDTAVYYCARDYDGYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 220 aa
DIQMTQSPSTLSASVGDRVTITCKSSQSLLNSSNQKNYLAWYQQKPGKAPKLLVYFASTRESGVPDRFIGSGSGTDFTLTISSLQPEDFATYFCHQHYSTPLTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 448 aa
QVQLVQSGAEVKKPGSSVKVSCKASGYTFTSYVMHWVRQAPGQGLEWIGYIYPYNDGTKYNEKFKGRVTITSDESTNTAYMELSSLRSEDTAVYYCAREEYGNYVRYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK